(19) United States
(12) Patent Application Publication
(10) Pub. No.: US 2005/0055014 A1
(43) Pub. Date: Mar. 10, 2005

(54) Methods for accelerated release of material from a reservoir device

(76) Inventors: Jonathan R. Coppeta, Windham, NH (US); John M. Maloney, Cambridge, MA (US); Benjamin F. Polito, Flagstaff, AZ (US); John T. Santini JR., North Chelmsford, MA (US); Norman F. Sheppard JR., Bedford, MA (US); Scott A. Uhland, Roslindale, MA (US)

Correspondence Address: SUTHERLAND ASBILL & BRENNAN LLP, 999 PEACHTREE STREET, N.E., ATLANTA, GA, 30309, US

(21) Appl. No.: 10/911,175

(22) Filed: Aug. 4, 2004

Related U.S. Application Data
(60) Provisional application No. 60/492,418, filed on Aug. 4, 2003.

(51) Int. Cl. A61K 9/22

(52) U.S. Cl. 604/890.1

(57) ABSTRACT

Methods and devices are provided for selective release or exposure of reservoir contents, such as a drug formulation or diagnostic reagent, sealed in a reservoir, e.g., a microreservoir. The devices comprise a substrate; one or more reservoirs located in and defined by the substrate; reservoir contents located inside the reservoirs; a reservoir cap or rupturable layer sealing an outlet of the reservoir; and means for disintegrating the reservoir cap or rupturing the rupturable layer; and means for accelerating the release of the reservoir contents from the reservoir through the outlet, or for enhancing diffusional mass transport of a material into or out of the reservoir. The means for accelerating can, for example, include a shape memory material. In a preferred embodiment, the device is adapted for implantation into a human or animal body and comprises an array of several discrete reservoir, each one being individually openable at a desired time.

### CROSS-REFERENCE TO RELATED APPLICATIONS

Benefit of U.S. Provisional Application No. 60/492,418, filed Aug. 4, 2003, is claimed. The application is incorporated herein by reference.

### BACKGROUND OF THE INVENTION

A variety of implantable drug delivery devices are known in the art for releasing drugs from small reservoirs. For example, microchip drug delivery devices are described in U.S. Pat. No. 5,797,898 and U.S. Pat. No. 6,527,762, to Santini et al. Several thermal techniques to accelerate drug release from reservoirs are described in U.S. Pat. No. 6,527,762, to Santini. U.S. Pat. No. 6,491,666 to Santini et al. describes, in one embodiment, a mechanical or physical process for rupturing reservoir caps to release drug from a reservoir for drug delivery; for example, an actuation pin is used to rupture a reservoir cap from a position external to the reservoir and substrate.

Drug release from non-implantable reservoir devices is also known. For example, U.S. Pat. No. 5,474,527 to Bettinger discloses a transdermal medication patch system wherein medication is dispensed internally by positive displacement from multiple reservoirs within the patch, using electric resistance heating elements to activate multiple heat-shrink polymer reservoirs to dispense fluidized medication into a common absorbent layer for transdermal passage.

Various devices and systems are described in the art for driving drugs or other chemicals from small reservoirs. Examples of these are found in U.S. Pat. No. 6,010,492 to Jacobsen et al., U.S. Pat. No. 5,196,002 to Hanover et al., and U.S. Pat. No. 5,167,625 to Jacobsen et al.

It would be desirable to provide new and improved systems and devices for accelerating release of reservoir contents from micro-reservoirs, particularly from implantable drug delivery devices.

### SUMMARY OF THE INVENTION

In one aspect, methods and devices are provided for the selective release or exposure of reservoir contents sealed in a reservoir. The devices comprise a substrate; one or more reservoirs located in and defined by the substrate; reservoir contents located inside the reservoirs; a reservoir cap or rupturable layer, sealing an outlet of the reservoir; and means for disintegrating the reservoir cap or rupturing the rupturable layer; and means for accelerating the release of the reservoir contents from the reservoir through the outlet, or for enhancing diffusional mass transport of a material into or out of the reservoir. In one embodiment, the means for accelerating is the means for rupturing the rupturable layer. In a preferred embodiment, the reservoirs are microreservoirs. In another preferred embodiment, the device is an implantable drug delivery device.

In one embodiment, the means for accelerating comprises a piston member driven by spring actuation or by volume expansion of an expansion agent. For example, the piston member can comprise a rigid plate or pin. In a particular embodiment, the means for accelerating release comprises a shape memory material, such as a shape memory alloy, a shape memory polymer, or combination thereof. For instance, the shape memory material can be in the form of a spring positioned at the end of the reservoir distal the opening, and the means for accelerating can further comprise a plate slidably positioned in the reservoir between the spring and the reservoir contents, the spring being actuatable to move the plate and reservoir contents towards the reservoir outlet to expel the reservoir contents from the reservoir. In another example, the expansion agent comprises a thermally expandable material disposed at the end of the reservoir distal the outlet. In one embodiment, the expansion agent and actuation means are, prior to expansion of the expandable material, separated from the reservoir contents by a layer of a hermetic material.

In another embodiment, the means for accelerating comprises a propellant disposed at the end of the reservoir distal the outlet, the propellant being actuatable to move the reservoir contents towards the reservoir outlet to expel the reservoir contents from the reservoir. For example, the propellant can react to generate a gas, the expansion of which displaces the reservoir contents from the reservoir. In one particular embodiment, the device further includes a flexible shell positioned in the reservoir between the propellant and the reservoir contents.

In yet another embodiment, the means for accelerating comprises electrodes and a voltage source that are capable of inducing the electroosmotic or iontophoretic transport of at least a portion of the reservoir contents. In a further embodiment, the means for enhancing diffusional mass transport includes a surface of the substrate having electrodes that can be biased to induce flow of a fluid across the surface adjacent the outlet.

In another embodiment, the means for accelerating or means for enhancing diffusional mass transport comprises a flexible membrane disposed in the outlet of the reservoir and a vibration source element. Optionally, the device can further include a resonating structure positioned outside of the reservoir.

In further embodiment, the means for accelerating comprises one or more resistive heating elements inside the reservoir or at the outlet, the resistive heating elements being operable to form bubbles in liquid reservoir contents.

In a still further embodiment, the means for accelerating comprises a magnetic field source and the reservoir contents comprises magnetic microparticles. For example, the reservoir contents can include a gel that expands upon subjection to a magnetic flux.

In a preferred embodiment, the device comprises a plurality of reservoirs and corresponding discrete reservoir caps. In one embodiment, the reservoir caps comprise a conductive material and the means for disintegrating comprising a source of electric current or potential and circuitry for disintegrating the reservoir caps. In one embodiment, the reservoir cap comprises a metal film. In various embodiments, the reservoir caps can be disintegrated by electrothermal ablation, electrochemical oxidation, by mechanical rupture, or by a thermally induced phase change.

In preferred embodiments, the reservoir contents comprise a drug. In other embodiments, the reservoir contents comprise a diagnostic agent or other reagent. In one particular embodiment, the reservoir contents comprise an enzyme or other catalyst.

In a preferred embodiment, the reservoir contents are hermetically sealed in the reservoir before release. In one embodiment, the substrate comprises silicon, a ceramic, a metal, or a combination thereof.

In another aspect, methods are provided for delivering to a site reservoir contents from a reservoir. The method comprises the steps of (i) providing at a site for delivery one of the devices described above; (ii) disintegrating the reservoir cap or rupturing the rupturable layer; and (iii) actuating the means for accelerating or the means for enhancing diffusional mass transport. In one embodiment, the reservoir cap disintegrates before actuation of the means for accelerating release. In another embodiment, the reservoir cap disintegration or rupture of the rupturable layer occurs partially or completely due to actuation of the acceleration means.

In yet another aspect, a method is provided for making an array of shape memory elements. The method comprises making a series of cuts into a sheet of a shape memory material to form a plurality of shape memory elements, each of which remains connected to said sheet following said making of series of cuts. In one embodiment, the shape memory material comprises a shape memory alloy. In one embodiment, each shape memory element comprises a spring. In one embodiment, the series of cuts are made by etching, laser machining, stamping, wire electrodischarge machining, or a combination thereof.

### BRIEF DESCRIPTION OF THE DRAWINGS

FIG. 1 is a cross-sectional view of one embodiment of a device for accelerated release, which comprises a shape memory alloy spring and a push plate for driving drug formulation out of a reservoir.

FIG. 2A is a plan view of an array of shape memory alloy springs fabricated in a single layer of a material. FIG. 2B and FIG. 2C are perspective views of one shape memory alloy spring before actuation and after actuation.

FIG. 3A is a cross-sectional view of one embodiment of a device for accelerated release using electroosmotic transport.

FIG. 3B is a cross-sectional view of one embodiment of a device, which uses electrodes biased to create flows across the substrate face, which can enhance diffusional mass transport of drug from a reservoir.

FIG. 4 is a cross-sectional view of one embodiment of a device for accelerated release, which comprises thermally expandable material and a piston for driving drug formulation out of a reservoir.

FIG. 5 is a cross-sectional view of one embodiment of a device using a flexible membrane and a vibration source to enhance diffusional and/or bulk mass transport properties of a fluid present in the reservoir.

FIG. 6 is a cross-sectional view of one embodiment of a device of FIG. 5 further comprising a resonating structure.

FIG. 7 is a plan view of one embodiment of an electrode structure for thermal ablation of a reservoir cap and for thermal cycling to cause fluid agitation in the reservoir following reservoir cap disintegration.

FIGS. 8A-B are cross-sectional views of two configurations of a device using resistive heaters for thermally form bubbles to agitate fluid near the bottom of the reservoir (FIG. 8A) or near the reservoir opening (FIG. 8B).

FIG. 9 is a cross-sectional view of one embodiment of a device for accelerated release, which comprises a propellant material and flexible shell holding the drug formulation in the reservoir, where a fast reaction of the propellant material produces a large volume expansion and pushes the protective shell, and with it the drug, out of the reservoir.

FIG. 10 is a cross-sectional view of one embodiment of a device for accelerating release using a magnetic field to drive a drug formulation containing magnetic particles.

### DETAILED DESCRIPTION OF THE INVENTION

Methods and devices have been developed to accelerate the release of reservoir contents, particularly a drug formulation, out of a reservoir device, such as an implantable drug delivery device. These methods and devices can readily be applied to the selective release or selective exposure of other reservoir contents, such as catalysts, reagents, secondary devices, or the like, stored/isolated in the reservoir. In a preferred embodiment, the device is adapted for implantation into a human or animal body and comprises an array of several discrete reservoir, each one being individually openable at a desired time.

The devices and methods described herein can provide very fast release of drug or other reservoir contents. Such embodiments may be useful, for example, in releasing drugs whose efficacy is dependent on a fast pharmacokinetic pulsatile profile. Additionally, it may be desirable to release drug quickly in response to an external event such as a heart attack. The present devices are particularly useful in applications where drug must be delivered more quickly than can be obtained using a diffusion-based device (e.g., a polymeric depot), and where the drug (or more particularly the device containing the drug) preferably is, or needs to already be, present in the patient prior to an incapacitating event (such as a heart attack, stroke, seizure) because there not be another person present to administer the drug or device at the critical time.

As used herein, the terms “accelerated release” and “accelerate the release” refers to an increase in the transport rate of drug out of the reservoir relative to the transport rate of the drug solely by diffusion down its own chemical potential gradient. The terms also refer to expelling reservoir contents that would not otherwise egress from an open reservoir, i.e., where no or negligible diffusion could occur, such as with certain solid drug formulations.

Certain of the accelerated release mechanisms described below can be readily adapted to accelerate or maximize transport (e.g., diffusion) of a fluid into the reservoir. For example, this can be used to direct a bodily fluid and an analyte therein into contact with a sensor located within the reservoir.

As used herein, the terms “comprise,” “comprising,” “include,” and “including” are intended to be open, non-limiting terms, unless the contrary is expressly indicated.

The Acceleration Methods and Devices

The release acceleration techniques can include the use of a push plate, pin or piston, driven by spring actuation or by various types of material expansion, electroosmotic transport, fluid agitation, controlled bubble formation, propellant actuation, or magnetic actuation.

Spring Actuation

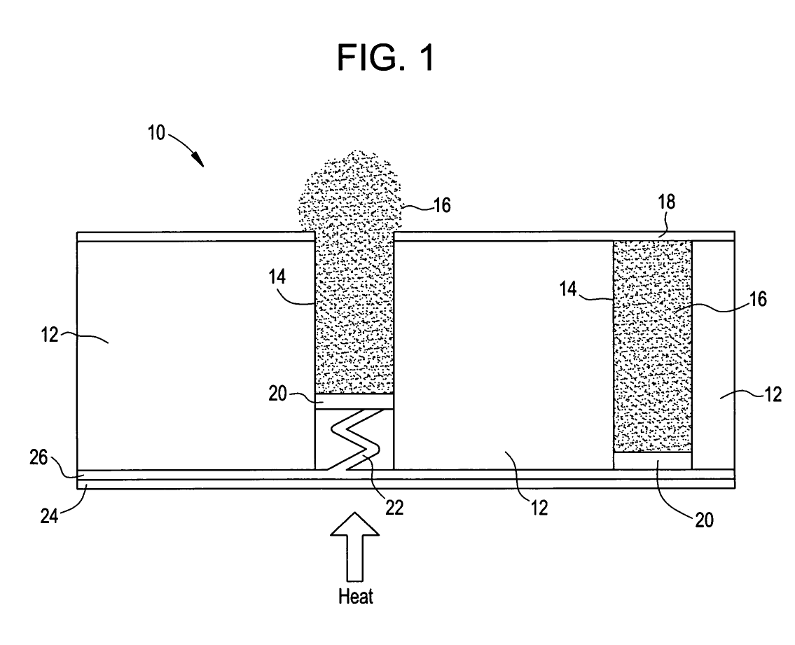

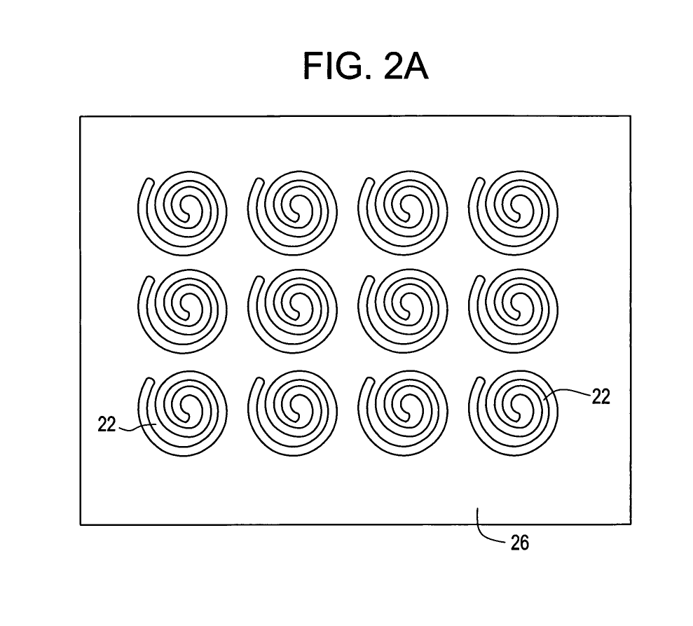

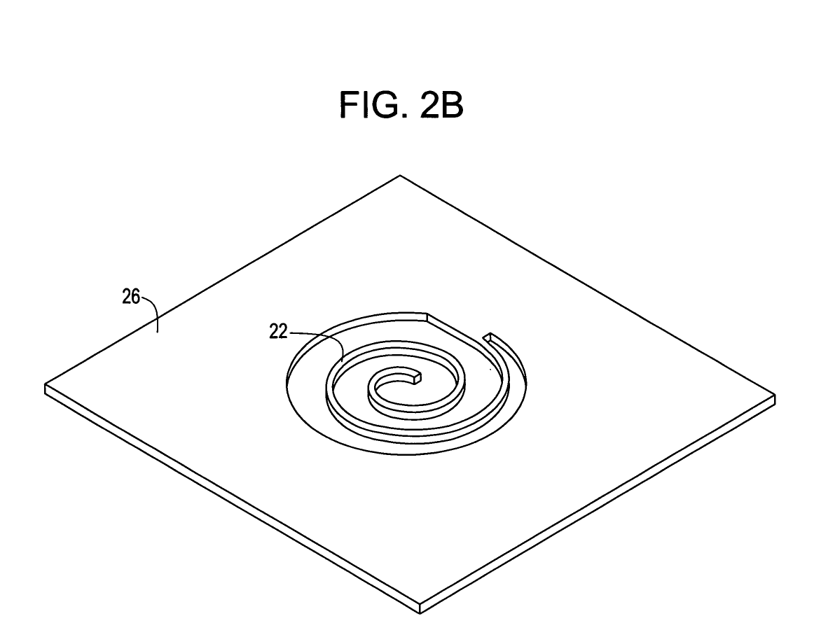

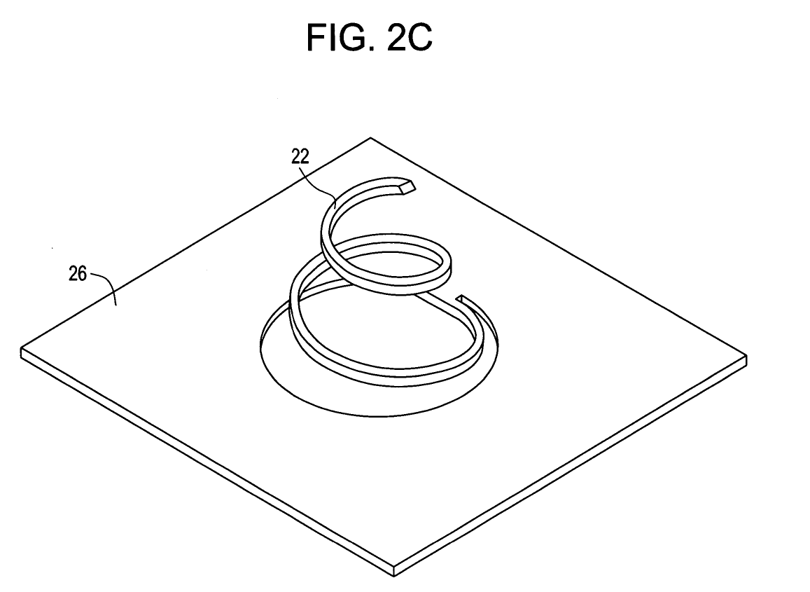

In one embodiment, a shape memory material, such as a shape memory alloy (SMA), a shape memory polymer, or a combination thereof, fashioned as a spring or lever is used to eject the contents from a reservoir upon thermal activation of the shape memory material. FIG. 1 illustrates one example of such an embodiment. Device 10 comprises a substrate 12 fabricated with an array of reservoirs 14 loaded with a push plate 20 and a drug formulation 16. One end of the reservoir is hermetically sealed with a discrete reservoir cap or continuous sealing layer 18. At the opposite end of the reservoir, where the push plate is located, the reservoir is closed by a sealing structure composed of a backing layer 24 and an SMA layer 26, which is bonded to the substrate/reservoirs such that each spring 22 fabricated in the SMA layer 26 is positioned next to the push plate 22 in the reservoir. FIG. 2A illustrates an array of twelve SMA springs formed in a layer of SMA, and FIGS. 2B-C show one of these springs before and after actuation.

Release (i.e., reservoir activation) is triggered by applying a local heat load to increase the temperature above the phase transition temperature of the SMA to actuate the spring. In one embodiment, the heat is locally generated by a microheater, attached directly or indirectly to the surface of the SMA layer. The microheaters can be made by patterning microheating elements, as known in the art.

The push plate is typically preferred, but could be omitted depending upon the particular reservoir contents, the particular design of the spring. The push plate is substantial rigid and can be formed of or coated with a biocompatible material. After the reservoir contents are ejected from the opening of the reservoir, the push plate can be designed to be retained in the reservoir, for example, by attachment to the spring or by having the opening include a stop feature, such as a tab or a narrowing of the reservoir, such that the push plate cannot be discharged. Alternatively, the push plate can be designed to permit its ejection. In such case, it may be desirable to form the push plate from a biodegradable polymer or other material.

Thermal isolation between springs may be accomplished by thinning or removing the SMA material between adjacent springs or by including an insulating material around each spring. Examples of insulating materials include oxides, nitrides, carbides, and other ceramics.

Rupturing of the reservoir cap can occur due to the force of the spring on the drug formulation or other reservoir contents pushing against/through the reservoir cap, or alternatively, the reservoir cap can be disintegrated or ruptured by an independent mechanism with subsequent coordinated spring actuation used to discharge the drug through the opening created by the other mechanism (e.g., thermal ablation, electrochemical oxidation, or thermally induced phase change of the reservoir cap). The reservoir contents can be one that is essentially incompressible, such as a liquid or dense solid mixture, or one that is slightly compressible, such as a porous (e.g., lyophilized) powder. In the latter case, actuation of the spring will cause the reservoir contents to compress some before it causes the reservoir cap to rupture.

In one variation, the SMA structure includes a lever or cantilever, in place of or in addition to a spring element, which applies the force to accelerate reservoir content release.

The spring or other actuator can be made from a variety of shape memory materials. The selection of an appropriate shape memory material depends on several factors, including the transformation temperature, mechanical properties, and biocompatibility. For an implanted medical device, the transformation temperature should be greater than about 45° C. to prevent inadvertent actuation, but should be as low as possible to minimize the heating required to effect the shape transformation. The transformation strain and other mechanical properties should be effective to overcome resistive forces and displace the push plate, so that the reservoir contents are driven out a particular reservoir at a useful rate and in a useful amount. If the actuator will be exposed to tissue, the material selected should be biocompatible or should be capable of being coated or encapsulated with a biocompatible material.

In one embodiment, the shape memory material comprises a shape memory alloy. A variety of shape memory alloys are known, including nickel-titanium alloys (e.g., NITINOL™) and alloys of copper (e.g., CuZnAl, CuAlNi). Nickel-titanium alloys are sufficiently biocompatible to find use in a variety of implanted medical devices such as stents.

In another embodiment, a shape memory polymer or block copolymer is used in place of or to augment the shape memory alloy. These may be attractive materials in certain application, because the polymers are light, high in shape recovery ability, easy to manipulate, and economical as compared with shape memory alloys. Examples of shape memory polymers are described for example in U.S. Pat. No. 6,388,043 and U.S. Pat. No. 6,160,084, as well as in Lendlien, et al., PNAS, 98(3):842-47 (2001)); Krulevitch, et al., J. Microelectromechanical Systems, 5(4):270-82 (1996); Lendlien et al., Angew. Chem. Int. Ed. 41:2034-57 (2002); and Gall et al., J. Microelectromechanical Systems, 13(3):472-83 (2004).

The springs or levers of the SMA structure can be made by a variety of methods. In one method, an array of SMA springs or levers are made in a single layer of material, e.g., by cutting springs into/out of a single sheet of SMA. For example, FIG. 2A illustrates a plan view of one embodiment of a sheet of SMA having an array of twelve spring-like structures fabricated therein. Methods suitable for creating the structures in the sheet include etching, laser machining, stamping, and wire electrodischarge machining. Once formed, the structures can be deformed to an extended configuration (out of plane, FIG. 2C) and annealed at a high temperature (typically, 450 to 550° C. for NITINOL). The annealing step programs the SMA to return to this configuration when heated beyond the phase transition temperature. The springs are then plastically deformed such that they are in-plane (FIG. 2B) with the sheet. The structures shown in FIGS. 2A-C represents one embodiment with the guiding design principle of large displacement monolithic springs.

In an alternative embodiment, the shape memory spring could be replaced with a bimetallic spring that also deforms under thermal load.

In yet another alternative embodiment, a spring inside the reservoir could be designed to pre-load the drug prior to activation. In this case, once the reservoir cap was removed (e.g., by using a technique for reservoir cap disintegration) or sufficiently weakened, then the reservoir contents would be forced out of the reservoir as the spring relaxed to its zero strain state. In other words, the reservoir contents are spring loaded in the reservoir, and upon removal of the reservoir cap, the compressed spring is unloaded to eject the reservoir contents from the reservoir. The spring could be made, for example, out of a compressible polymer, a metal, or a shape memory material.

Electroosmotic Transport

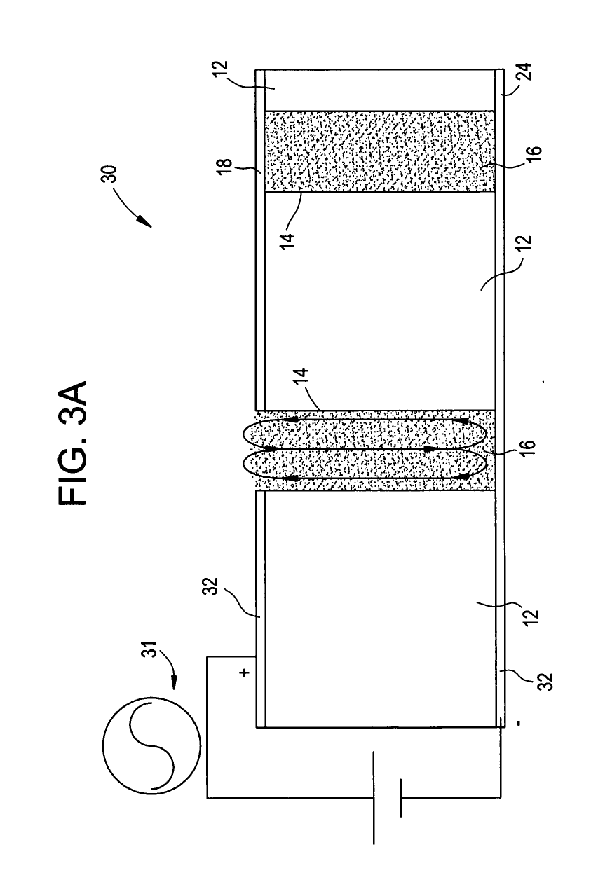

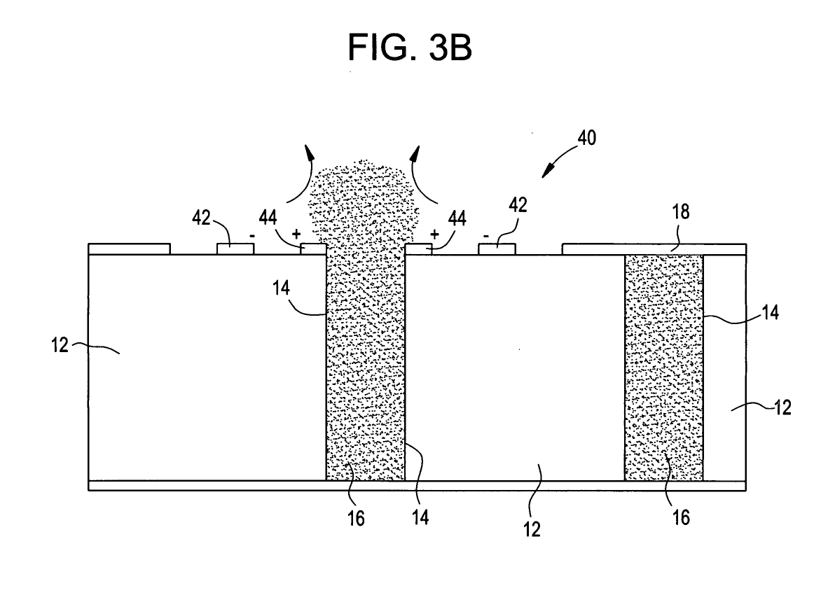

In another technique, an electrical potential is used to accelerate release from reservoirs using the known phenomenon of electroosmotic or iontophoretic transport. One embodiment of a device adapted to use this acceleration technique is illustrated in FIG. 3. Device 30 includes a substrate 12 having a plurality of reservoirs 14 loaded with a drug formulation 16. Two reservoirs are shown: The one on the right is sealed, with the release opening covered by reservoir cap 18, and the one on the left has been opened. A voltage generating means 31 is electrically connected to electrodes 32 at both ends/surfaces of the reservoir, to disintegrate the reservoir cap (e.g., using an electrocorrosion process, as described in U.S. Pat. No. 5,797,898 to Santini, et al.). The electrodes/conductive leads 32 on the front (release side) of the substrate that are used to deliver the energy to remove the reservoir cap are biased relative to the back of the substrate. Either an AC or a DC voltage can be used. In many cases, the resulting electric field creates a force on charged particles in the reservoir, causing those particles to move toward the oppositely charged electrode. Because one end of the reservoir is closed by backing layer 24, a recirculating flow will develop inside the reservoir enhancing mixing with body fluid at the reservoir outlet. Transport rates of 0.5 mm/s have been reported in circular capillaries for field strengths of 300 V/cm (Lenne, et al., Flow Profiles and Directionality in Microcapillaries Measured by Fluorescence Correlation Spectroscopy, Single Mol., 3:194-200 (2002)). This technique could be applied even if the drug itself is not charged provided one species in the reservoir is charged and is mobile. The bulk flow induced by the movement of the charged species will essentially drag or carry the non-charged drug out of the reservoir.

Another embodiment is illustrated in FIG. 3B. It shows device 40 which includes substrate 12 in which reservoirs 14 are disposed. The reservoirs are loaded with drug formulation 16. Two reservoirs are shown: The one on the right is sealed, with the release opening covered by reservoir cap 18, and the one on the left has been opened. The surface of the substrate on the release side includes patterns of electrodes 42 and 44 which can be biased to create flows across the substrate face, which in turn can enhance diffusional mass transport of drug from the reservoir by keeping the drug concentration at the reservoir opening at or near zero (i.e., infinite sink conditions). This results in the greatest possible concentration gradient or diffusional driving force.

These electroosmotic transport techniques can be used with essentially any reservoir opening technique, to enhance release or exposure of the reservoir contents. For example, the reservoir cap could be opened by a thermal activation method and then a voltage can be applied to facilitate mixing of the drug with the biological fluids at a site of implantation in a patient.

Rupture and Accelerated Release Using Mechanical Actuation

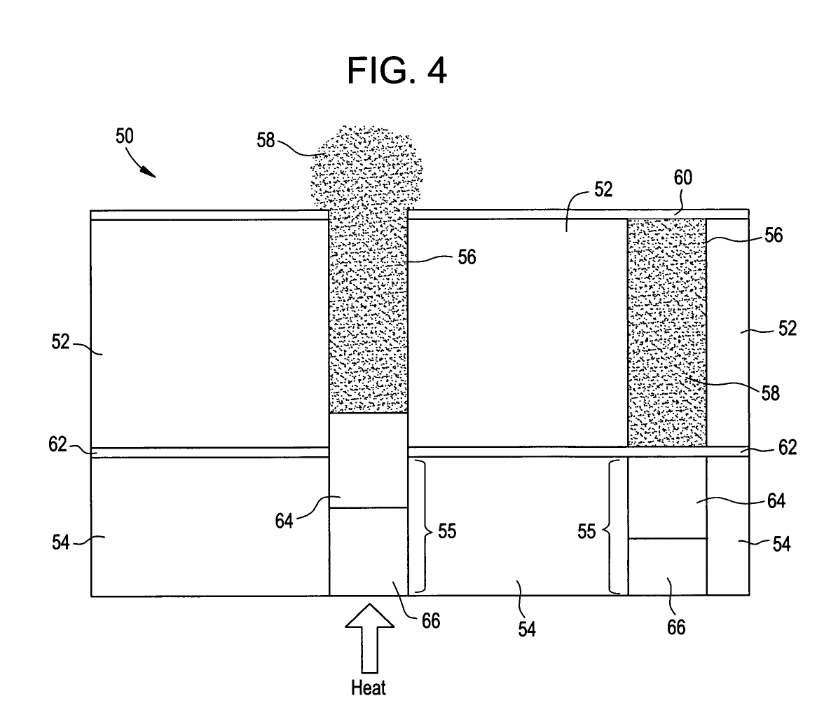

In one embodiment, an actuation pin (or piston or plate) is adapted for accelerating release of reservoir contents from a reservoir, by pushing into and through one end of a hermetically sealed reservoir to cause a second end of the reservoir to rupture, expelling the contents in the process. An example of one embodiment of such a device is illustrated in FIG. 4. The device 50 includes a primary substrate 52 having reservoirs 56 containing a drug formulation 58.

Two reservoirs are shown: The one on the right is in its hermetically sealed state, with the release opening covered by reservoir cap 60, and the one on the left has been opened. The sealed reservoir is sealed on the side of the primary substrate distal the release side by a hermetic sealing layer 62. A secondary substrate 54 is bonded to the hermetic layer and primary substrate. The secondary substrate 54 includes actuation reservoirs 55 that are aligned with each drug-loaded reservoir 56 in the primary substrate 52. Each actuation reservoir 55 contains and serves as a guide path for an actuation pin 64 contained therein. Prior to actuation, the actuation pin 64 is positioned adjacent the hermetic layer 62 beneath each drug reservoir (as illustrated in the right reservoir of FIG. 4). Beneath each pin is a thermally expandable material or propellant 66. Applying heat to the thermally expandable material 66 causes the actuation pin 64 to be forced through the hermetic layer 62 and into the drug reservoir 56, creating enough pressure on the drug to rupture the reservoir cap 60 and expel the drug out from the reservoir (as illustrated in the left reservoir of FIG. 4).

A variety of expandable materials and expansion triggers can be utilized. For example, the expandable material may be a thermally expandable wax, a foaming agent, a propellant, or a thermally generated bubble (which could be generated as described below).

The actuation pin may be formed of, for example, a polymer (e.g., a polytetrafluoroethylene) or inorganic material such as a metal or ceramic (e.g., oxides, nitrides, carbides, and the like). It preferably is inert, biocompatible, and non-reactive with the drug formulation.

For an implantable medical device, the actuation system (i.e., which includes the actuation pin and the expandable material) should be formed of a biocompatible material, or it should be contained in, encapsulated within, or coated with a biocompatible material. In one embodiment, the opening at the release-end of the reservoir (i.e., the opening through which the drug is released) is slightly smaller than the largest cross-sectional dimension of the actuation pin so that the actuation pin will be retained in the reservoir after expelling all of the reservoir contents, so that the pin is not released into the patient's body. In another embodiment, the actual pin, the expandable material, or both, are biodegradable or bioerodible (e.g., formed of a polyester, such as PLGA, or a polyhydroxyalkanoate or a polyanhydride), so that when/if they are released they can degrade into harmless constituents that can be used by or excreted from the body.

In another embodiment, the actuation system may be incorporated within the drug reservoir if so desired (i.e., eliminating the need for a secondary substrate or second rupture point in walls defining the reservoir).

In an alternative embodiment, the actuation pin may be displaced using actuation methods other than thermal expansion. For example, a magnetic field created by a solenoid or other method could be used to repel (i.e., drive) a magnetic pin.

Fluid Agitation

In another method, an agitation means is used to enhance diffusional and/or bulk mass transport properties of a fluid present in the reservoir, to accelerate and/or enhance release or exposure of the reservoir contents. By agitating the fluid, drug egress from the reservoir can be increased.

In one embodiment, the means for accelerating comprises a flexible membrane disposed in the outlet of the reservoir and a vibration source. Here a vibration source is anything that causes a local periodic displacement in proximity to a reservoir or causes the entire device to experience a periodic displacement. Periodic displacement (e.g., frequencies of 1 Hz to several megahertz depending on the geometries involved) of the flexible membrane is used to increase local mixing at the reservoir outlet, thus accelerating release of the drug. Accelerated release may be created if convective flow is achieved; if not, then maximum diffusion based on a zero concentration at the release opening may be achieved with this embodiment.

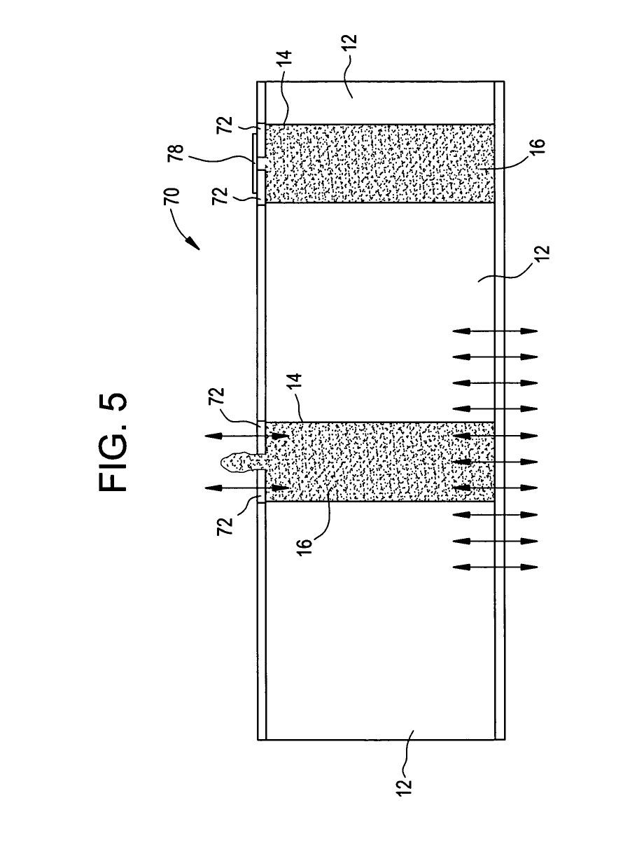

One example of such a device is illustrated in FIG. 5. Device 70 includes substrate 12 in which an array of reservoirs 14 is defined. The reservoirs are loaded with drug formulation 16. A flexible membrane 72 is disposed in the outlet (i.e., the release-side opening of the reservoir) of the reservoirs. Two reservoirs are shown: The one on the right is sealed, with the release opening covered by reservoir cap 78, and the one on the left has been opened. A vibration source (not shown) is adapted to cause the flexible membrane to be periodically displaced. In operation, the reservoir cap can be disintegrated or ruptured using essentially any reservoir opening technique (e.g., electrochemical oxidation, thermal activation, thermal ablation, or mechanical rupture).

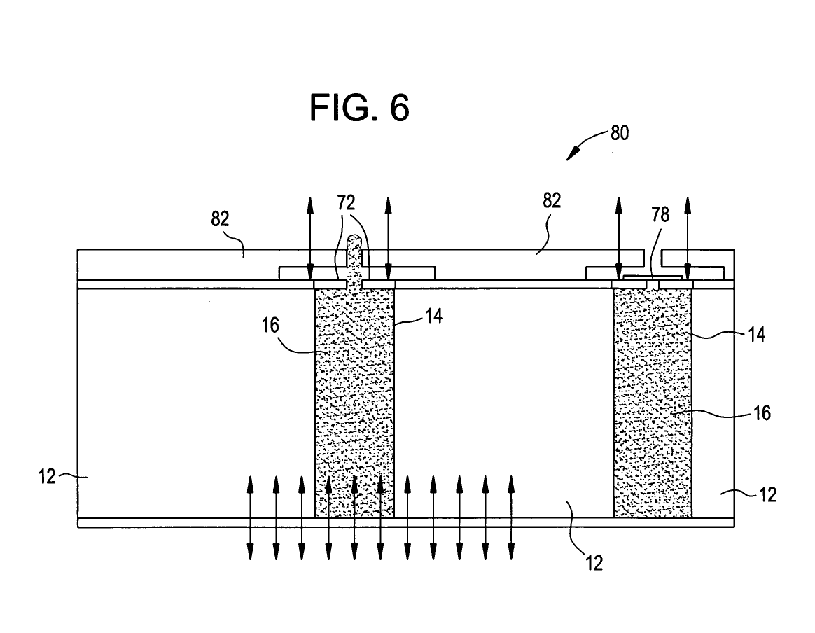

Optionally, a resonating structure can be positioned outside of the reservoir. One such embodiment is illustrated in FIG. 6. Device 80 is identical to device 70 (shown in FIG. 5) except that device 80 includes a resonating structure 82 near the reservoir outlet. The resonating structure should provide fluid agitation near the selected reservoir outlet while minimizing the displacement of nearby intact (i.e., un-activated) reservoir caps.

The flexible membrane can be made, for example, out of a dielectric material such as silicon nitride, silicon dioxides, silicon oxynitride, or any another impermeable material such as silicon, or a thin metal film that will not plastically deform during vibration/flexing. The membranes can be made to vibrate by vibrating the entire device at the resonant frequency of the membrane. In one embodiment, the vibration is achieved by coupling a PZT element to the substrate. Careful design of the membrane and reservoir cap geometry may make it possible to minimize the vibration of membranes where the reservoir cap has not been activated. It may be undesirable to directly displace the reservoir cap prior to activation. The PZT element also may be activated only on or near a particular reservoir by applying a voltage or current to selected areas of the PZT element.

The ability of the resonating structure to resonate depends, in part, on damping conditions created by the fluid environment in which the device is operated. The resonating structure typically is bonded to the substrate. The resonating structure can be made from a variety of materials. Examples of suitable materials include polymers, metals, dielectrics, and other organic material. In one embodiment, an SOI wafer may be used if it is desired to make the resonating structures monolithic. The resonating structure may be a vibrating membrane with a hole in the center, a cantilever, or any other structure that will maximize displacement. Both the structure and material selection can be useful in designing a structure that will resonate at frequencies far from the reservoir cap resonant frequency.

Yet another approach to agitating fluid at the reservoir outlet is to induce/effect mechanical displacement of the membrane in the direction normal to the plane of the membrane. This creates a displacement of the reservoir opening relative to the fluid. One method to accomplish this displacement is to periodically heat the reservoir membrane. Thermal expansion in the plane of the membrane will cause it to periodically deflect in the direction normal to the membrane. For example, this could be accomplished by placing resistive heating elements electrically in parallel with the reservoir cap. In one embodiment, the circuit intended to accelerate release by piezoelectric or electrothermal means is electrically separate from the reservoir cap or any circuit used to open the reservoir.

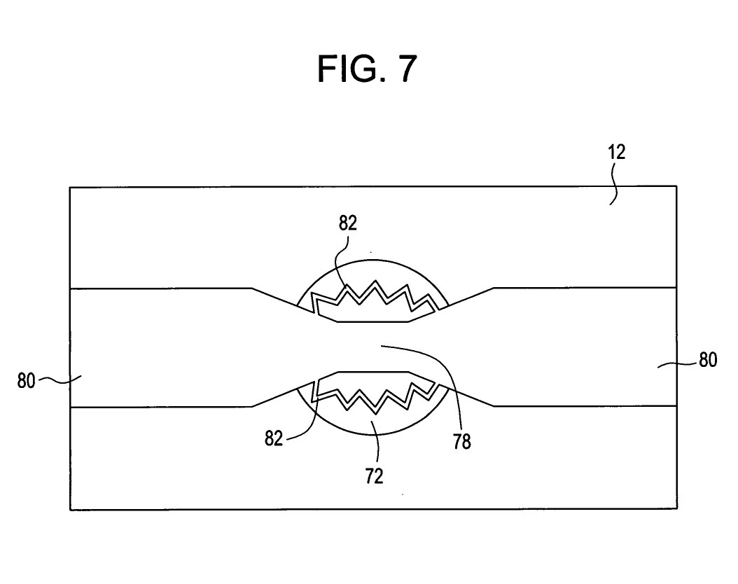

In another embodiment, such as in the case where the reservoir is opened by passing current through the reservoir cap, the circuit intended to accelerate release is electrically in parallel with the reservoir cap. One example of this displacement mechanism is illustrated in FIG. 7, which could be incorporated in device 70 (shown in FIG. 5). This figure shows substrate 12, flexible membrane 72, and reservoir cap 78, which is connected to electrical traces 80 and to resistive heaters 82 in parallel with the reservoir cap could cause deflection of a membrane by thermally induced strain. The resistance of these heaters is desirably large (>10 times) when compared to the resistance of the reservoir cap to ensure that most of the current passes through the reservoir cap until it is purposefully ablated to open the reservoir. The resistance of the resistive elements is designed such that most of the current passes through the reservoir cap until it is purposefully electrically ablated to open the reservoir as described in U.S. Patent Application Publication No. 2004/0121486 A1. Once the reservoir cap is opened, a periodic current transmits through the resistive elements causing the membrane to flex resulting in a pumping motion to eject fluid from the reservoir.

In one variation, bimetallic heating elements are provided on the membrane such that the displacement is induced by the deflection of the heating elements rather than or in addition to the thermal expansion of the membrane material itself. In another variation, the membrane is provided with an electrical trace running across a surface of the membrane, which trace contributes to or dominates the buckling of the membrane.

In another embodiment, the resistive elements are used to periodically displace a resonating structure separate from the membrane as in FIG. 6, except that the whole device need not vibrate. In this case, the vibration source is the resistive heaters causing membrane buckling.

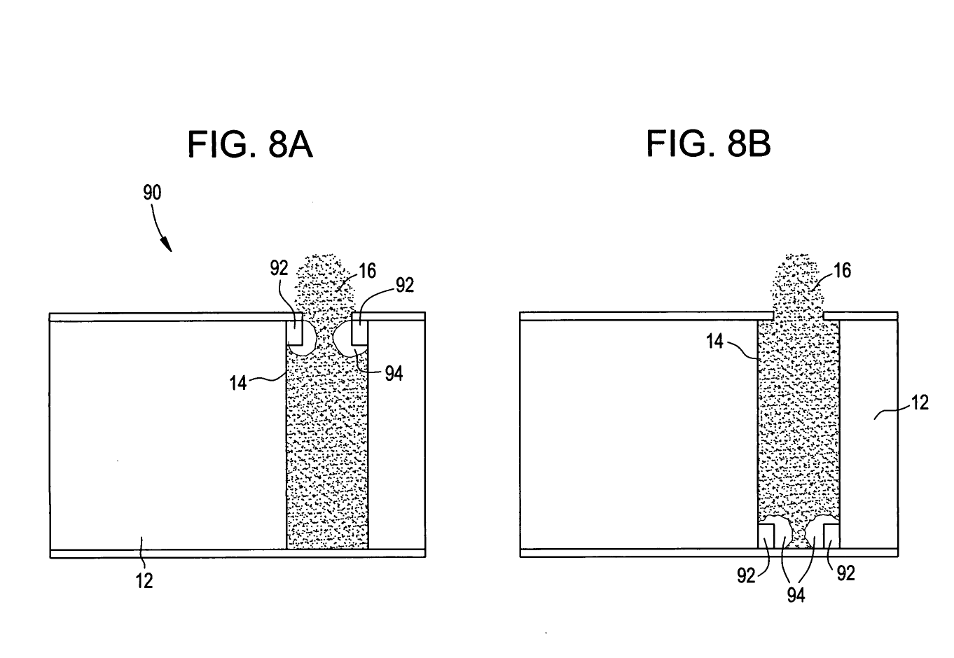

In still another embodiment, periodic bubble formation is employed to agitate fluid in the reservoir or near the reservoir opening. In one variation, resistive heating elements (like those shown in FIG. 7) are adapted to heat the fluid at/near the reservoir opening/outlet to periodically form bubbles to cause fluid mixing at the reservoir outlet. FIGS. 8A-B show two other heater configurations that create thermal bubbles to mix the reservoir fluid. In FIGS. 8A-B, a device 90 includes a substrate 12 which has reservoir 14 disposed therein. The reservoirs contain a fluid 16, e.g., a drug formulation, and one or more resistive heaters 92. The figures show bubbles 94 being formed around the heaters and enhancing or accelerating release of the reservoir contents from the outlet of the reservoir. In FIG. 8A, the heaters are located in the reservoir adjacent the outlet. In FIG. 8B, the heaters are located in the bottom of the reservoir, distal the outlet.

Reservoir caps for any of these embodiments may be opened by a variety of means. In one example, pressure created by thermal bubble formation is used to rupture the reservoir cap, to open it. In another example, the reservoir cap is disintegrated by electrical ablation or electrochemical oxidation. In another embodiment, and where the device is in contact with an electrolyte such as saline or serum, electrolysis is used to form gas bubbles to cause mixing at the reservoir outlet.

In another embodiment, gentle thermal gradients are used to create buoyancy driven flow (natural convection). In this approach, the density variation with temperature under the influence of gravity will cause fluid motion. For example, one or more heaters can be placed within a reservoir in a similar configuration to the thermal bubble approach described above. The direction of the buoyant flow will depend on the orientation of the reservoir with respect to gravity. Therefore, if the device is not fixed in one orientation, the release profiles may vary to some degree.

Chemical Potential

In another technique, the chemical formulation of the reservoir contents (e.g., drug formulation) is modified to increase the transport rate of water into the reservoir or to increase the transport rate of the drug out of the reservoir. For instance, a hygroscopic formulation, such as one containing a polymer, such as a polyethylene glycol (PEG), can be used to rapidly draw water into the reservoir upon release, creating a bulk flow that enhances mixing. In another embodiment, a polymer formulation can be used that undergoes a volume expansion upon mixing with water. That is, water solvation causes the polymer to increase its radius of gyration or unfold and occupy a greater volume than in a purely formulated form. Heats of mixing may also be used to create natural convection as described above. In yet another embodiment, the formulation could be contained in an evacuated reservoir to pull water into the reservoir upon reservoir cap disintegration.

Propellant Actuation

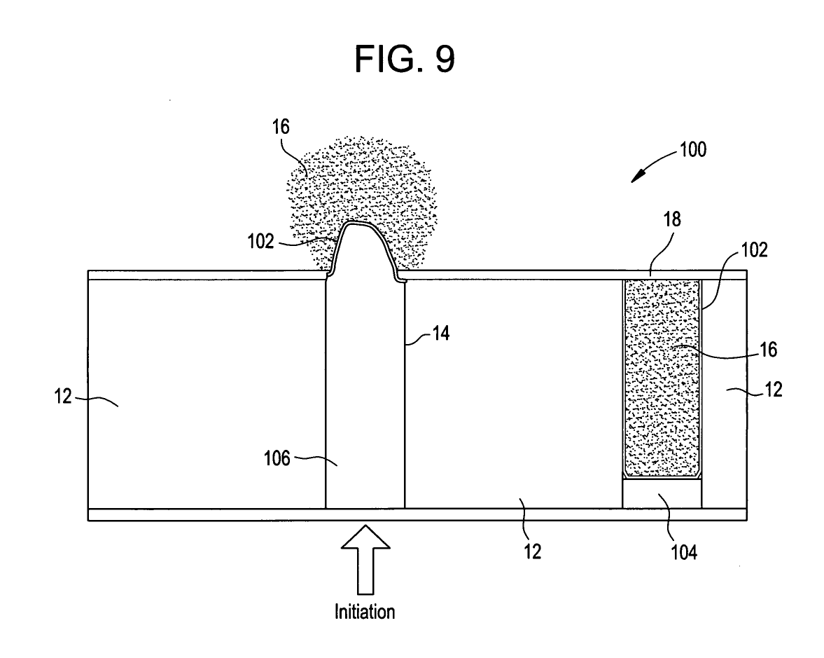

In another technique, a propellant is used to expel reservoir contents from a reservoir. One embodiment of such a device is illustrated in FIG. 9. Device 100 includes substrate 12 in which an array of reservoirs 14 is defined. Two reservoirs are shown: The one on the right is sealed, with the release opening covered by reservoir cap 18, and the one on the left has been opened and the reservoir contents expelled. The sealed reservoir contains a drug formulation 16 which is disposed inside a protective shell 102, which can be a flexible bag. The bottom of the reservoir includes a propellant material 104 directly beneath the drug formulation-loaded shell. Upon initiation/activation of the propellant (which can be by application of heat or a spark or the like), a fast exothermic reaction proceeds. As shown in the figure by the opened reservoir, the reaction product gases 106 produce a large volume expansion and push the protective shell, and with it the drug, out of the reservoir.

Examples of propellants include nanocrystalline Si, nano-particles of Ti, sodium bicarbonate (thermal decomposition), and elemental sodium (mixing with water). Other examples of suitable propellant materials are known in the art.

The protective shell preferably is a thin, flexible, strong material, formed for example, from a polymer. It should protect the drug against thermal degradation or chemical reaction with the propellant.

In one embodiment, a reservoir cap covers the reservoir opening and is disintegrated or ruptured by a separate mechanism (e.g., thermal ablation) prior to propellant actuation. U.S. Pat. No. 5,167,625 to Jacobsen et al. discloses other sealing/opening structures and mechanisms for expelling reservoir contents.

Magnetic Actuation

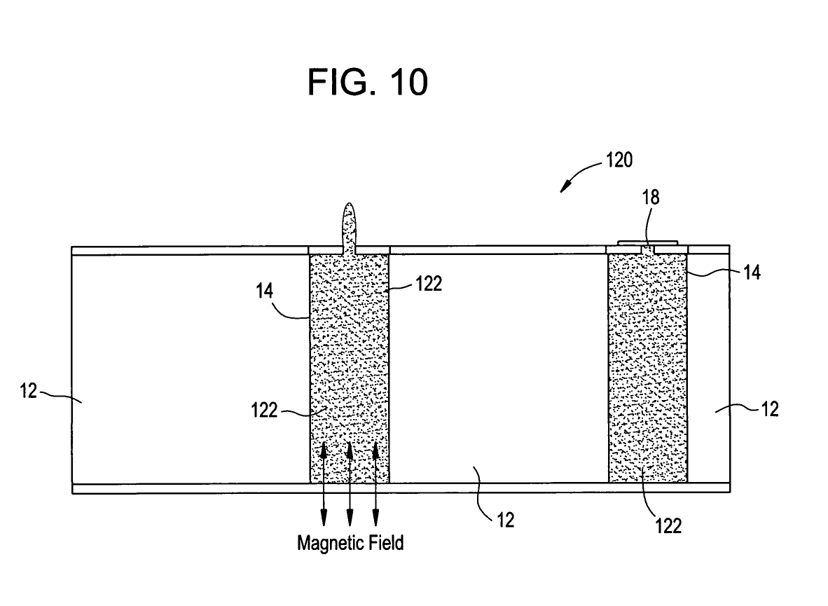

In yet another technique, the means for accelerating release utilizes a magnetic field. One embodiment of such a device is illustrated in FIG. 10. Device 120 includes a substrate 12 having a plurality of reservoirs 14 loaded with a drug formulation 122 which includes a gel interdispersed with micron-sized magnetic particles. Two reservoirs are shown: The one on the right is sealed, with the release opening covered by reservoir cap 18, and the one on the left has been opened. In operation, a magnetic flux is applied to cause the gel to expand, forcing the drug formulation 122 out of the reservoir.

In one embodiment, the drug formulation comprises a drug-containing gel interdispersed with magnetic particles (e.g., a ferrogel, which is a chemically cross-linked polymer network swollen by a ferrofluid, for example polyvinyl alcohol crosslinked with glutardaldehyde). In various embodiments, the magnetic particles are magnetic, paramagnetic, or superparamagnetic particles. This could be implemented as a preload on the drug before release or as a release mechanism. Strains up to 40% have been reported by Zrinyi M, Barsi L, Buki A at http://www.kfki.hu/˜cheminfo/hun/olvaso/zrinyi/polymgel.html. See also Polymer Gels Netw. 5(5):415-27 (1997); Ach-Models Chem 134 (2-3):155-67 (1997); Magy Kem Foly 103(9):401-10 (1997).

Rupturing of the reservoir cap can occur due to the force created by the magnetically induced expansion of the gel pushing against/through the reservoir cap, or alternatively, the reservoir cap can be disintegrated or ruptured by an independent mechanism with subsequent, coordinated magnetic actuation used to discharge the drug through the opening created by the other mechanism.

In another variation, instead of using a gel, 1 μm (or smaller or larger) diameter magnetic spheres could be incorporated in the drug formulation. Application of a non-uniform magnetic field will impart a force on the magnetic particles creating a convective flow to drag the drug out of the reservoir. Magnetic fields could be created on the whole substrate at once by placing a voice coil on top of the reservoir array, or each reservoir could have a current carrying conductor in close proximity to generate local magnetic fields. A uniform magnetic field may also be superimposed on the spatially varying magnetic field to enhance the dipole magnetic moment of the particles. Velocities of 1 mm/s have been reported using magnetic microspheres in a microfluidic channel (Rida, et al., Planar Coil-Based Microsystem for the Long-Range Transport of Magnetic Beads, 12^{th} Int'l Conf. on Solid State Sensors, Actuators, and Microsystems, Boston, Jun. 8-12, 2003, Vol. 1, pp. 292-95). See also Edelman & Langer, Biomaterials 14(8):621-26 (1993); Edelman, et al., J. Biomed. Mater. Res. 19:67-83 (1985); Edelman, et al., J. Biomed. Mater. Res. 21(3):339-53 (1987).

Other Details of the Reservoir Device

The particular design of present devices depends in part of the intended application, the desired size and number of discrete reservoir contents to be isolated, the operating environment, and the selected mechanism for opening the reservoirs and/or triggering acceleration of release. Examples of reservoir devices that can be adapted to use the present acceleration methods are described in U.S. Pat. No. 5,797,898, U.S. Pat. No. 6,551,838, U.S. Pat. No. 6,527,762, as well as in U.S. patent application publications No. 2002/0099359 and No. 2003/0010808, which are incorporated herein by reference. In one embodiment, the device is a medical device, such as an implantable drug delivery device or an implantable sensor device.

Substrate and Reservoirs

In one embodiment, the reservoir device comprises a body portion, i.e., a substrate, that includes one or more reservoirs for hermetically containing reservoir contents. That is, the substrate is the structural body (e.g., part of a device) in which the reservoirs are formed, e.g., it contains the etched, machined, or molded reservoirs. A reservoir is a well, a container, or a cavity. In a preferred embodiment, the device includes a plurality of the reservoirs located in discrete positions across at least one surface of the body portion.

Reservoirs can be fabricated in a structural body portion using any suitable fabrication technique known in the art. Representative fabrication techniques include MEMS fabrication processes or other micromachining processes, various drilling techniques (e.g., laser, mechanical, and ultrasonic drilling), and build-up techniques, such as LTCC (low temperature co-fired ceramics). The surface of the reservoir optionally can be treated or coated to alter one or more properties of the surface. Examples of such properties include hydrophilicity/hydrophobicity, wetting properties (surface energies, contact angles, etc.), surface roughness, electrical charge, release characteristics, and the like. MEMS methods, micromolding, micromachining, and microfabrication techniques known in the art can be used to fabricate the substrate/reservoirs from a variety of materials. Numerous other methods known in the art can also be used to form the reservoirs. See, for example, U.S. Pat. No. 6,123,861 and U.S. Patent Application Publication No. 2002/0107470.

In various embodiments, the body portion of the containment device comprises silicon, a metal, a ceramic, a polymer, or a combination thereof. Examples of suitable substrate materials include metals, ceramics, semiconductors, glasses, and degradable and non-degradable polymers. Preferably each reservoir is formed of hermetic materials (e.g., metals, silicon, glasses, ceramics) and is hermetically sealed by a reservoir cap. In a preferred embodiment, the substrate material is biocompatible and suitable for long-term implantation into a patient. In a preferred embodiment, the substrate is formed of one or more hermetic materials. The substrate, or portions thereof, may be coated, encapsulated, or otherwise contained in a hermetic biocompatible material (e.g., inert ceramics, titanium, and the like) before use. If the substrate material is not biocompatible, then it can be coated with, encapsulated, or otherwise contained in a biocompatible material, such as poly(ethylene glycol), polytetrafluoroethylene-like materials, diamond-like carbon, inert ceramics, titanium, and the like, before use. In one embodiment, the substrate is hermetic, that is impermeable (at least during the time of use of the reservoir device) to the molecules to be delivered and to surrounding gases or fluids (e.g., water, blood, electrolytes or other solutions). In another embodiment, the substrate is made of a material that degrades or dissolves over a defined period of time into biocompatible components. Examples of such materials include biocompatible polymers, such as poly(lactic acid)s, poly(glycolic acid)s, and poly(lactic-co-glycolic acid)s, as well as degradable poly(anhydride-co-imides).

The substrate can have a range of shapes or shaped surfaces. It can, for example, have a planar or curved surface, which for example could be shaped to conform to an attachment surface. In various embodiments, the substrate or the containment device is in the form of a chip, a circular or ovoid disk, a tube, a sphere, or a stent. The substrate can be flexible or rigid.

The substrate may consist of only one material, or may be a composite or multi-laminate material, that is, composed of several layers of the same or different substrate materials that are bonded together. Substrate portions (as in FIG. 1) can be, for example, silicon or another micromachined substrate or combination of micromachined substrates such as silicon and Pyrex glass, e.g., as described in U.S. patent application Ser. No. 09/665,303 or U.S. Pat. No. 6,527,762. In another embodiment, the substrate comprises multiple silicon wafers bonded together. In yet another embodiment, the substrate comprises a low-temperature co-fired ceramic (LTCC). In one embodiment, the body portion is the support for a microchip device. In one example, this substrate is formed of silicon.

Total substrate thickness and reservoir volume can be increased by bonding or attaching wafers or layers of substrate materials together. The device thickness may affect the volume of each reservoir and/or may affect the maximum number of reservoirs that can be incorporated onto a substrate. The size and number of substrates and reservoirs can be selected to accommodate the quantity and volume of reservoir contents needed for a particular application, manufacturing limitations, and/or total device size limitations to be suitable for implantation into a patient, preferably using minimally invasive procedures.

The substrate can have one, two, or preferably many, reservoirs. In various embodiments, tens, hundreds, or thousands of reservoirs are arrayed across the substrate. For instance, one embodiment of an implantable drug delivery device includes between 250 and 750 reservoirs, where each reservoir contains a single dose of a drug for release. In one sensing embodiment, the number of reservoirs in the device is determined by the operation life of the individual sensors. For example, a one-year implantable glucose monitoring device having individual sensors that remain functional for 30 days after exposure to the body would contain at least 12 reservoirs (assuming one sensor per reservoir). In another sensor embodiment, the distance between the sensor surface and the reservoir opening means is minimized, preferably only a few microns. In this case, the volume of the reservoir is primarily determined by the surface area of the sensor. For example, the electrodes of a typical enzymatic glucose sensor may occupy a space that is 400 μm by 800 μm.

In one embodiment, the reservoirs are microreservoirs. As used herein, the term “microreservoir” refers to a concave-shaped solid structure suitable for releasably containing a material, wherein the structure is of a size and shape suitable for filling with a microquantity of the material, which comprises a drug. In one embodiment, the microreservoir has a volume equal to or less than 500 μL (e.g., less than 250 μL, less than 100 μL, less than 50 μL, less than 25 μL, less than 10 μL, etc.) and greater than about 1 nL (e.g., greater than 5 nL, greater than 10 nL, greater than about 25 nL, greater than about 50 nL, greater than about 1 μL, etc.). The shape and dimensions of the microreservoir can be selected to maximize or minimize contact area between the drug material and the surrounding surface of the microreservoir.

As used herein, the term “microquantity” refers to small volumes between 1 nL and 10 μL. In one embodiment, the microquantity is between 1 nL and 1 μL. In another embodiment, the microquantity is between 10 nL and 500 nL.

In other embodiments, the reservoirs are larger than microreservoirs and can contain a quantity of drug formulation larger than a microquantity. For example, the volume of each reservoir can be greater than 10 μL (e.g., at least 20 μL, at least 50 μL, at least 100 μL, at least 250 μL, etc.) and less than 1,000 μL (e.g., less than 900 μL, less than 750 μL, less than 500 μL, less than 300 μL, etc.). These may be referred to as macro-reservoirs and macro-quantities, respectively. Unless explicitly indicated to be limited to either micro- or macro-scale volumes/quantities, the term “reservoir” is intended to include both.

In a preferred embodiment, the materials and construction of the devices provide that the reservoirs are hermetically sealed. As used herein, the term “hermetic” refers to preventing undesirable chemical ingress or egress into or out of one or more compartments of the device, particularly the device reservoirs, over the useful life of the device, using a seal composed of materials, such as ceramics, glasses, and metals, which are essentially impermeable to chemicals and biological fluids such as water, oxygen, and carbon dioxide.

Reservoir Contents

The reservoir contents are essentially any object or material that needs to be isolated (e.g., protected from) the environment outside of the reservoir until a selected point in time, when its release or exposure is desired. In various embodiments, the reservoir contents comprise (a quantity of) chemical molecules, a secondary device, or a combination thereof.

Proper functioning of certain reservoir contents, such as a catalyst or sensor, generally does not require release from the reservoir; rather their intended function, e.g., catalysis or sensing, occurs upon exposure of the reservoir contents to the environment outside of the reservoir after opening of the reservoir cap. Thus, the catalyst molecules or sensing component can be released or can remain immobilized within the open reservoir. Other reservoir contents such as drug molecules often may need to be released from the reservoir in order to pass from the device and be delivered to a site in vivo to exert a therapeutic effect on a patient. However, the drug molecules may be retained within the reservoirs for certain in vitro applications.

Chemical Molecules

The reservoir contents can include essentially any natural or synthetic, organic or inorganic molecules or mixtures thereof. The molecules may be in essentially any form, such as a pure solid or liquid, a gel or hydrogel, a solution, an emulsion, a slurry, or a suspension. The molecules of interest may be mixed with other materials to control or enhance the rate and/or time of release from an opened reservoir. In various embodiments, the molecules may be in the form of solid mixtures, including amorphous and crystalline mixed powders, monolithic solid mixtures, lyophilized powders, and solid interpenetrating networks. In other embodiments, the molecules are in liquid-comprising forms, such as solutions, emulsions, colloidal suspensions, slurries, or gel mixtures such as hydrogels.

In a preferred embodiment, the reservoir contents comprise a drug formulation. The drug formulation is a composition that comprises a drug. As used herein, the term “drug” includes any therapeutic or prophylactic agent (e.g., an active pharmaceutical ingredient or API). In one embodiment, the drug is provided in a solid form, particularly for purposes of maintaining or extending the stability of the drug over a commercially and medically useful time, e.g., during storage in a drug delivery device until the drug needs to be administered. The solid drug matrix may be in pure form or in the form of solid particles of another material in which the drug is contained, suspended, or dispersed. In one embodiment, the drug is formulated with an excipient material that is useful for accelerating release, e.g., a water-swellable material that can aid in pushing the drug out of the reservoir and through any tissue capsule over the reservoir.

The drug can comprise small molecules, large (i.e., macro-) molecules, or a combination thereof. In one embodiment, the large molecule drug is a protein or a peptide. In various other embodiments, the drug can be selected from amino acids, vaccines, antiviral agents, gene delivery vectors, interleukin inhibitors, immunomodulators, neurotropic factors, neuroprotective agents, antineoplastic agents, chemotherapeutic agents, polysaccharides, anti-coagulants (e.g., LMWH, pentasaccharides), antibiotics (e.g., immunosuppressants), analgesic agents, and vitamins. In one embodiment, the drug is a protein. Examples of suitable types of proteins include, glycoproteins, enzymes (e.g., proteolytic enzymes), hormones or other analogs (e.g., LHRH, steroids, corticosteroids, growth factors), antibodies (e.g., anti-VEGF antibodies, tumor necrosis factor inhibitors), cytokines (e.g., α-, β-, or γ-interferons), interleukins (e.g., IL-2, IL-10), and diabetes/obesity-related therapeutics (e.g., insulin, exenatide, PYY, GLP-1 and its analogs). In one embodiment, the drug is a gonadotropin-releasing (LHRH) hormone analog, such as leuprolide. In another exemplary embodiment, the drug comprises parathyroid hormone, such as a human parathyroid hormone or its analogs, e.g., hPTH(1-84) or hPTH(1-34). In a further embodiment, the drug is selected from nucleosides, nucleotides, and analogs and conjugates thereof. In yet another embodiment, the drug comprises a peptide with natriuretic activity, such as atrial natriuretic peptide (ANP), B-type (or brain) natriuretic peptide (BNP), C-type natriuretic peptide (CNP), or dendroaspis natriuretic peptide (DNP). In still another embodiment, the drug is selected from diuretics, vasodilators, inotropic agents, anti-arrhythmic agents, Ca⁺ channel blocking agents, anti-adrenergics/sympatholytics, and renin angiotensin system antagonists. In one embodiment, the drug is a VEGF inhibitor, VEGF antibody, VEGF antibody fragment, or another anti-angiogenic agent. Examples include an aptamer, such as MACUGEN™ (Pfizer/Eyetech) (pegaptanib sodium)) or LUCENTIS™ (Genetech/Novartis) (rhuFab VEGF, or ranibizumab), which could be used in the prevention of choroidal neovascularization (useful in the treatment of age-related macular degeneration or diabetic retinopathy). In yet a further embodiment, the drug is a prostaglandin, a prostacyclin, or another drug effective in the treatment of peripheral vascular disease.

In one embodiment, the device delivers one or more drugs known in the art for use in pain management. Examples include lidocaine and fentanyl.

In another embodiment, the drug is an angiogenic agent, such as VEGF. In a further embodiment, the drug is an anti-inflammatory, such as dexamethasone. In one embodiment, a device includes both angiogenic agents and anti-inflammatory agents.

The reservoirs in one device can include a single drug or a combination of two or more drugs, and can further include one or more pharmaceutically acceptable carriers. Two or more drugs can be stored together and released from the same one or more reservoirs or they can each be stored in and released from different reservoirs.

For in vitro applications, the chemical molecules can be any of a wide range of molecules where the controlled release of a small (milligram to nanogram) amount of one or more molecules is required, for example, in the fields of analytic chemistry or medical diagnostics. Molecules can be effective as pH buffering agents, diagnostic reagents, and reagents in complex reactions such as the polymerase chain reaction or other nucleic acid amplification procedures. In various other embodiments, the molecules to be released are fragrances or scents, dyes or other coloring agents, sweeteners or other concentrated flavoring agents, or a variety of other compounds. In yet other embodiments, the reservoirs contain immobilized molecules. Examples include any chemical species which can be involved in a reaction, including reagents, catalysts (e.g., enzymes, metals, and zeolites), proteins, nucleic acids, polysaccharides, cells, and polymers, as well as organic or inorganic molecules which can function as a diagnostic agent.

The drug or other molecules for release can be dispersed in a matrix material, to control the rate of release. This matrix material can be a “release system,” as described in U.S. Pat. No. 5,797,898, the degradation, dissolution, or diffusion properties of which can provide a method for controlling the release rate of the chemical molecules.

Particularly for drugs, the release system may include one or more pharmaceutical excipients. The release system may provide a temporally modulated release profile (e.g., pulsatile release) when time variation in plasma levels is desired or a more continuous or consistent release profile when a constant plasma level as needed to enhance a therapeutic effect, for example. Pulsatile release can be achieved from an individual reservoir, from a plurality of reservoirs, or a combination thereof. For example, where each reservoir provides only a single pulse, multiple pulses (i.e. pulsatile release) are achieved by temporally staggering the single pulse release from each of several reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of molecules through it over an extended period. In addition, continuous release can be approximated by releasing several pulses of molecules in rapid succession (“digital” release). The active release systems described herein can be used alone or on combination with passive release systems, for example, as described in U.S. Pat. No. 5,797,898. For example, the reservoir cap can be removed by active means to expose a passive release system, or a given substrate can include both passive and active release reservoirs.

In one embodiment, the drug formulation within a reservoir comprises layers of drug and non-drug material. After the active release mechanism has exposed the reservoir contents, the multiple layers provide multiple pulses of drug release due to intervening layers of non-drug.

Secondary Devices

In another embodiment, the reservoir contents include a secondary device, alone or in combination with chemical molecules. As used herein, unless explicitly indicated otherwise, the term “secondary device” includes any device or a component thereof that can be located in a reservoir. In one embodiment, the secondary device is a sensor or sensing component thereof. As used herein, a “sensing component” includes a component utilized in measuring or analyzing the presence, absence, or change in a chemical or ionic species, energy, or one or more physical properties (e.g., pH, pressure) at a site. Types of sensors include biosensors, chemical sensors, physical sensors, or optical sensors. Secondary devices are further described in U.S. Pat. No. 6,551,838. In one embodiment, the sensor is a pressure sensor. See, e.g., U.S. Pat. No. 6,221,024, and U.S. Pat. No. 6,237,398, and U.S. Patent Application Publication No. 2004/0073137. Examples of sensing components include components utilized in measuring or analyzing the presence, absence, or change in a drug, chemical, or ionic species, energy (or light), or one or more physical properties (e.g., pH, pressure) at a site.

In one variation, the reservoir includes a sensor and a reagent, and the reagent desirably is released quickly. The released reagent is involved in a reaction, and then the sensor senses a reaction product or condition.

In one embodiment, a device is provided for implantation in a patient (e.g., a human or other mammal) and the reservoir contents comprise at least one sensor indicative of a physiological condition in the patient. For example, the sensor could monitor the concentration of glucose, urea, calcium, or a hormone present in the blood, plasma, interstitial fluid, vitreous humor, or other bodily fluid of the patient.

Several options exist for receiving and analyzing data obtained with secondary devices located within the primary device, which can be a microchip device or another device. Devices may be controlled by local microprocessors or remote control. Biosensor information may provide input to the controller to determine the time and type of activation automatically, with human intervention, or a combination thereof. For example, the operation of the device can be controlled by an on-board (i.e., within the package) microprocessor. The output signal from the device, after conditioning by suitable circuitry if needed, will be acquired by the microprocessor. After analysis and processing, the output signal can be stored in a writeable computer memory chip, and/or can be sent (e.g., wirelessly) to a remote location away from the microchip. Power can be supplied to the microchip system locally by a battery or remotely by wireless transmission. See, e.g., U.S. Patent Application Publication No. 2002/0072784.

In one embodiment, a device is provided having reservoir contents that include drug molecules for release and a sensor/sensing component. For example, the sensor or sensing component can be located in a reservoir or can be attached to the device substrate. The sensor can operably communicate with the device, e.g., through a microprocessor, to control or modify the drug release variables, including dosage amount and frequency, time of release, effective rate of release, selection of drug or drug combination, and the like. The sensor or sensing component detects (or not) the species or property at the site of in vivo implantation and further may relay a signal to the microprocessor used for controlling release from the device. Such a signal could provide feedback on and/or finely control the release of a drug. In another embodiment, the device includes one or more biosensors (which may be sealed in reservoirs until needed for use) that are capable of detecting and/or measuring signals within the body of a patient.

In one variation, an implantable medical device includes reservoirs comprising sensor, sealed as described herein, and a signal from the sensor is transmitted (by any number of means, including hardwire or telemetry) to a separate drug delivery device, which could be a wearable (i.e., external) or internal pump, the signal being used in the control of the dosing of the drug.

As used herein, the term “biosensor” includes sensing devices that transduce the chemical potential of an analyte of interest into an electrical signal, as well as electrodes that measure electrical signals directly or indirectly (e.g., by converting a mechanical or thermal energy into an electrical signal). For example, the biosensor may measure intrinsic electrical signals (EKG, EEG, or other neural signals), pressure, temperature, pH, or mechanical loads on tissue structures at various in vivo locations. The electrical signal from the biosensor can then be measured, for example by a microprocessor/controller, which then can transmit the information to a remote controller, another local controller, or both. For example, the system can be used to relay or record information on the patient's vital signs or the implant environment, such as drug concentration.

In one embodiment, the device contains one or more sensors for use in glucose monitoring and insulin control. Information from the sensor could be used to actively control insulin release from the same device or from a separate insulin delivery device (e.g., a conventional insulin pump, either an externally worn version or an implanted version). Other embodiments could sense other analytes and delivery other types of drugs in a similar fashion.

Reservoir Caps and Control Means Therefor

The reservoir device includes a rupturable layer or reservoir cap. For example, in devices having an array of multiple individual reservoirs, the reservoirs can be cover by one or more layers of a rupturable material. This rupturable layer may be present as pieces covering two or more of the reservoirs, or the rupturable layer can be present as one continuous layer covering all the reservoirs. Alternatively, each reservoir can be covered by a discrete reservoir cap, where each reservoir cap corresponds to a single reservoir. Each reservoir cap is separately actuable, i.e., it can be selectively and individually disintegrated or ruptured. In one embodiment, combinations of these layers and reservoir caps are used.

In preferred embodiments, the reservoir cap is selectively disintegrated. As used herein, the term “disintegrate” includes degrading, dissolving, rupturing, fracturing or some other form of mechanical failure, as well as a loss of structural integrity due to a chemical reaction (e.g., electrochemical degradation) or phase change (e.g., melting) in response to a change in temperature, unless a specific one of these mechanisms is indicated.

In one embodiment, the reservoir device includes reservoir caps and the hardware, electrical components, and software needed to control and deliver electric energy from a power source to selected reservoir(s) for actuation, e.g., reservoir opening. The reservoir cap or part of it (e.g., one layer of it) can be disintegrated or permeabilized by a separate means (such as thermal ablation or electrochemical oxidation or thermal rupture) before or simultaneously with actuation of the acceleration means. In one embodiment, the reservoir cap comprises at least two layers, one of which is disintegrated by thermal ablation and another of which is ruptured from which by action of an acceleration means described above, wherein release or exposure of the reservoir contents does not occur until both the thermal ablation means and the acceleration means have been activated.

As used herein, the term “reservoir cap” includes a membrane or other structure suitable for separating the contents of a reservoir from the environment outside of the reservoir. It generally is self-supporting across the reservoir opening, although caps having additional structures to provide mechanical support to the cap can be fabricated. See, e.g., U.S. Patent Application Publication Nos. 2002/0183721 A1. Reservoir caps can be made using MEMS or other techniques and designed/fabricated to open to the external environment upon activation by any of a number of methods, including those taught in U.S. Pat. No. 6,527,762, U.S. Pat. No. 5,797,898, and U.S. Patent Application Publication No. 2004/0121486 A1.

The reservoir cap could include any material that can be disintegrated or permeabilized in response to an applied stimulus (e.g., electric field or current, magnetic field, change in pH, or by thermal, chemical, electrochemical, or mechanical means).

In one embodiment, the reservoir cap comprises a metal film, or other conductive material, that is disintegrated by electrothermal ablation as described in U.S. Patent Application Publication No. 2004/0121486 A1. Other reservoir cap opening and release control methods are described in U.S. Pat. No. 5,797,898, U.S. Pat. No. 6,527,762, and U.S. Pat. No. 6,491,666, U.S. Patent Application Publication Nos. 2002/0107470 A1, 2002/0072784 A1, 2002/0138067 A1, 2002/0151776 A1, 2002/0099359 A1, 2002/0187260 A1, and 2003/0010808 A1; PCT WO 2004/022033 A2; PCT WO 2004/026281; and U.S. Pat. Nos. 5,797,898; 6,123,861; and 6,527,762, all of which are incorporated by reference herein.

In one embodiment, the disintegration is by an electro-thermal ablation technique, as described in U.S. Patent Application Publication No. 2004/0121486 A1. While not wishing to be bound by any theory, this thermal ablation is believed to cause the removal of the reservoir cap by a thermally-induced melting, a thermally-induced mechanical shock/rupture or a combination thereof. For example, the reservoir cap can be formed of a conductive material, such as a metal film, through which an electrical current can be passed to electrothermally ablate it, as described in U.S. Patent Application Publication No. 2004/0121486 A1. Representative examples of suitable reservoir cap materials include gold, copper, aluminum, silver, platinum, titanium, palladium, various alloys (e.g., Au—Si, Au—Ge, Pt—Ir, Ni—Ti, Pt—Si, SS 304, SS 316), and silicon doped with an impurity to increase electrical conductivity, as known in the art. In one embodiment, the reservoir cap is in the form of a thin metal film. In one example, the reservoir cap is part of a multiple layer structure, For instance, the reservoir cap can be made of multiple metal layers, such as a multi-layer/laminate structure of platinum/titanium/platinum. The reservoir cap is operably (i.e., electrically) connected to an electrical input lead and to an electrical output lead, to facilitate flow of an electrical current through the reservoir cap. When an effective amount of an electrical current is applied through the leads and reservoir cap, the temperature of the reservoir cap is locally increased due to resistive heating, and the heat generated within the reservoir cap increases the temperature sufficiently to cause the reservoir cap to be electrothermally ablated and ruptured.

In another specific embodiment, the “disintegration” is by an electrochemical activation technique, such as described in U.S. Pat. No. 5,797,898. For example, the reservoir cap can be a thin metal film impermeable to the surrounding environment (e.g., body fluids or another chloride containing solution). It is activated/opened by applying an electric potential to the metal reservoir cap, which is then oxidized and disintegrated by an electrochemical reaction. Examples of suitable reservoir cap materials include gold, silver, copper, and zinc.

In yet another specific embodiment, the “disintegration” is by an thermal activation technique, such as described in U.S. Pat. No. 6,527,762 or U.S. Pat. No. 6,669,683. For example, the reservoir cap can be heated (e.g., using resistive heating) to cause the reservoir cap to melt and be displaced from the reservoir to open it. This latter variation could be used, for example, with reservoir caps formed of a metal or a non-metal material, e.g., a polymer. In yet another variation, the reservoir cap is formed of a polymer or other material that undergoes a temperature-dependent change in permeability such that upon heating to a pre-selected temperature, the reservoir is rendered permeable to the drug and bodily fluids to permit the drug to be released from the reservoir through the reservoir cap.

It is also possible that the reservoir cap is designed to disintegrate by passive mechanisms prior to actuation of the acceleration means. For example, the reservoir cap could be formed from a material or mixture of materials that degrade, dissolve, or disintegrate over time, or that do not degrade, dissolve, or disintegrate, but are permeable or become permeable to molecules or energy. For instance, the reservoir cap can be formed of one or more polymers or copolymers or blends. Characteristics (such as polymer, degree of crosslinking, or polymer thickness) can be different for each reservoir cap to provide different times of release/exposure of reservoir contents. For example, any combination of can be modified to obtain a specific release time or rate. In other embodiments, non-polymeric materials such as porous forms of metals, semiconductors, and ceramics are used. Passive semiconductor reservoir cap materials include nanoporous or microporous silicon membranes.

Uses of the Release Techniques

The devices and methods described herein can be used to facilitate release or exposure of a variety of reservoir contents in a wide variety of applications. Preferred applications include the controlled delivery of one or more drugs, biosensing, or a combination thereof.

In one embodiment, a device is used to deliver a drug systemically to a patient in need thereof. In another embodiment, the construction and placement of the microchip in a patient enables the local or regional release of drugs that may be too potent for systemic delivery of an effective dose. The reservoir contents in one reservoir or in one device can include a single drug or a combination of two or more drugs, and the reservoir contents can further include pharmaceutically acceptable carriers. In some embodiments, the present devices for accelerated release are incorporated into a drug pump, a stent, or an inhaler or other pulmonary drug delivery device.

In one particular embodiment, the reservoir contents comprises a drug formulation comprising parathyroid hormone, such as a human parathyroid hormone, e.g., hPTH(1-84) or hPTH(1-34). It is important to deliver this drug in a pulsatile manner.

In a preferred embodiment, the sealed reservoir device is part of an implantable medical device. The implantable medical device can take a wide variety of forms and be used in a variety of therapeutic and/or diagnostic applications. Examples include implantable controlled drug delivery devices, drug pumps (such as an implantable osmotic or mechanical pump), drug-eluting stents, and combinations thereof. In one embodiment, the device includes releases a drug formulation, is implanted into a patient (such as a human or other vertebrate animal) using standard surgical or minimally-invasive implantation techniques, and then the reservoirs are opened on a schedule determined by the type of drug therapy prescribed by the physician. In another example, the device is adapted for transdermal drug delivery.

In another embodiment, the device includes (i) active release reservoirs containing sensors. For example, the device could include a plurality of sensors isolated until the time their exposure to the environment is desired. The environment could be in vitro or in vivo, depending upon the particular application and device. In one embodiment, the sensor is a biosensor, and the reservoirs are opened as needed (depending, for example, upon fouling of the sensor) or as dictated by a predetermined schedule. In one embodiment, the sealed reservoirs contain pressure sensors.

In other embodiments, the reservoirs described herein are incorporated into a variety of other devices from which it is desirable to quickly release chemical molecules or other reservoir contents. The devices have numerous in vivo, in vitro, and commercial diagnostic applications. The devices are capable of delivering precisely metered quantities of molecules and thus are useful for in vitro applications, such as analytical chemistry and medical diagnostics, as well as biological applications such as the delivery of factors to cell cultures. In still other non-medical applications, the devices are used to control release of fragrances, dyes, or other useful chemicals. Methods of using and operating the devices are further described in U.S. Pat. Nos. 5,797,898; 6,527,762; 6,491,666; and 6,551,838, and U.S. Patent Application Publications 2002/0183721, 2003/0100865, 2002/0099359, 2004/0082937, 2004/0127942, 2004/0121486, 2004/0106914, and 2004/0106953, all of which are incorporated by reference herein.

Publications cited herein are incorporated by reference. Modifications and variations of the methods and devices described herein will be obvious to those skilled in the art from the foregoing detailed description. Such modifications and variations are intended to come within the scope of the appended claims.

1. A device for the selective release or exposure of reservoir contents sealed in a reservoir comprising:
a substrate;
one or more reservoirs located in and defined by the substrate;
reservoir contents located inside the reservoirs;
a reservoir cap or rupturable layer sealing an outlet of the reservoir; and
means for disintegrating the reservoir cap or rupturing the rupturable layer; and
means for accelerating the release of the reservoir contents from the reservoir through the outlet, or for enhancing diffusional mass transport of a material into or out of the reservoir.

2. The device of claim 1, comprising means for accelerating the release of the reservoir contents, wherein the means for accelerating comprises a piston member driven by spring actuation or by volume expansion of an expansion agent.

3. The device of claim 2, wherein the piston member comprises a rigid plate or pin.

4. The device of claim 2, wherein the means for accelerating release comprises a shape memory material.

5. The device of claim 4, wherein the shape memory material comprises a shape memory alloy.

6. The device of claim 4, wherein the shape memory material comprises a shape memory polymer.

7. The device of claim 4, wherein the shape memory material is in the form of a spring positioned at the end of the reservoir distal the opening, and the means for accelerating further comprises a plate slidably positioned in the reservoir between the spring and the reservoir contents, the spring being actuatable to move the plate and reservoir contents towards the reservoir outlet to expel the reservoir contents from the reservoir.

8. The device of claim 2, wherein the expansion agent comprises a thermally expandable material disposed at the end of the reservoir distal the outlet.

9. The device of claim 1, wherein the means for accelerating comprises a propellant disposed at the end of the reservoir distal the outlet, the propellant being actuatable to move the reservoir contents towards the reservoir outlet to expel the reservoir contents from the reservoir.

10. The device of claim 9, wherein the propellant reacts to generate a gas, the expansion of which displaces the reservoir contents.

11. The device of claim 9, further comprising a flexible shell positioned in the reservoir between the propellant and the reservoir contents.

12. The device of claim 8, wherein the expansion agent and actuation means are, prior to expansion of the expandable material, separated from the reservoir contents by a layer of a hermetic material.

13. The device of claim 1, wherein the means for accelerating comprises electrodes and a voltage source which are capable of inducing the electroosmotic or iontophoretic transport of at least a portion of the reservoir contents.

14. The device of claim 1, comprising means for enhancing diffusional mass transport, wherein a surface of the substrate comprises electrodes which can be biased to induce flow of a fluid across the surface adjacent the outlet.

15. The device of claim 1, wherein the means for accelerating or means for enhancing diffusional mass transport comprises a flexible membrane disposed in the outlet of the reservoir and a vibration source element.

16. The device of claim 15, further comprising a resonating structure positioned outside of the reservoir.

17. The device of claim 1, wherein the means for accelerating comprises one or more resistive heating elements inside the reservoir or at the outlet, the resistive heating elements being operable to form bubbles in liquid reservoir contents.

18. The device of claim 1, wherein the means for accelerating comprises a magnetic field source and the reservoir contents comprises magnetic microparticles.

19. The device of claim 18, wherein the reservoir contents further comprises a gel which expands upon subjection to a magnetic flux.

20. The device of claim 2, wherein the means for accelerating is the means for rupturing the rupturable layer.

21. The device of claim 1, which comprises a plurality of reservoirs and corresponding discrete reservoir caps.

22. The device of claim 21, which comprises a means for disintegrating the reservoir caps, wherein the reservoir caps comprise a conductive material and the means for disintegrating comprising a source of electric current or potential and circuitry for disintegrating the reservoir caps.

23. The device of claim 21, wherein the reservoir cap disintegrates by electrothermal ablation.

24. The device of claim 21, wherein the reservoir caps disintegrate by electrochemical oxidation.

25. The device of claim 21, wherein the reservoir caps disintegrate by mechanical rupture.

26. The device of claim 21, wherein the reservoir caps disintegrate by a thermally induced phase change.

27. The device of claim 1, wherein the reservoir contents comprises a drug.

28. The device of claim 1, wherein the reservoir contents comprises a diagnostic agent or other reagent.

29. The device of claim 1, wherein the reservoir contents comprises an enzyme or other catalyst.

30. The device of claim 1, wherein the reservoirs are microreservoirs.

31. The device of claim 1, wherein the substrate comprises silicon, a ceramic, a metal, or a combination thereof.

32. The device of claim 1, wherein the reservoir cap comprises a metal film.

33. The device of claim 1, wherein the reservoir cap disintegrates before actuation of the means for accelerating release.

34. The device of claim 1, wherein the reservoir cap disintegration or rupture of the rupturable layer occurs partially or completely due to actuation of the acceleration means.

35. The device of claim 1, wherein the reservoir contents are hermetically sealed in the reservoir before release.

36. The device of claim 1, which is an implantable drug delivery device.

37. A method for delivering to a site reservoir contents from a reservoir comprising:
providing at a site for delivery the device of claim 1;
disintegrating the reservoir cap or rupturing the rupturable layer; and
actuating the means for accelerating or the means for enhancing diffusional mass transport.

38. A method for making an array of shape memory elements, comprising:
making a series of cuts into a sheet of a shape memory material to form a plurality of shape memory elements, each of which remains connected to said sheet following said making of series of cuts.

39. The method of claim 38, wherein the shape memory material comprises a shape memory alloy.

40. The method of claim 38, wherein each shape memory element comprises a spring.

41. The method of claim 38, wherein said series of cuts are made by etching, laser machining, stamping, wire electrodischarge machining, or a combination thereof.
